(19) United States
(12) Patent Application Publication
(10) Pub. No.: US 2007/0104795 A1
(43) Pub. Date: May 10, 2007

(54) Multiphase active ingredient formulation

(75) Inventors: Daniel Rudhardt, Koln (DE); Frank Ridder, Kurten (DE); Stefan Hofmann, Langenfeld (DE)

Correspondence Address: NORRIS, MCLAUGHLIN & MARCUS, P.A., 875 THIRD AVE, 18TH FLOOR, NEW YORK, NY, 10022, US

(73) Assignee: Bayer Technology Services GmbH, Leverkusen (DE)

(21) Appl. No.: 10/583,302

(22) PCT Filed: Dec. 15, 2004
(86) PCT No.: PCT/EP04/14261

§ 371 Date: Oct. 25, 2006

(30) Foreign Application Priority Data
Dec. 19, 2003 (DE) 103 59 792.1

(51) Int. Cl. A61K 9/50; A61K 9/16

(52) U.S. Cl. 424/490

(57) ABSTRACT

The invention relates to a formulation having a plurality of phases containing active ingredients. Said formulation is characterised in that: it contains a first, innermost, finely distributed phase (I) consisting of active ingredients or an active ingredient solution, preferably some of the phase particles being surrounded by a barrier sheath (M); the formulation has a second, intermediate phase (II) that serves as a dispersant for the first, inner phase (I) and can also be dissolved in the active ingredient; and the formulation has a third, outer phase (III) that serves as a dispersant for the second intermediate phase and can, in turn, be dissolved in the active ingredient and/or can be in the form of solid particles that can, in turn, be surrounded by a barrier sheath. In this way, a plurality of biological active ingredients can be produced in the different phases in different concentrations and with a respectively controlled release rate in a single formulation. According to the invention, a mechanical stabilisation is achieved by solidifying the intermediate phase (II) enabling the formulation to be stable for a longer period of time even once it has been applied to a surface. Furthermore, by selecting a suitable solvent for the outer phase, the formulations limit the release of the active ingredient from the innermost phase, and the capsules are not emptied and can thus be stored for a longer period of time.

The invention relates to an active-ingredient-containing formulation with a plurality of active-ingredient-containing phases.

A large number of active ingredients are liquids or are in the form of a substance dissolved in liquid. A way was sought to control the release over time of such an active ingredient in a targeted manner after it had been applied to a surface which is in contact with a gas space. In particular, a way was sought here of delaying the release of the active ingredient, of controlling the release rate of the active ingredient, of providing chemically or biologically incompatible active ingredients in one formulation and/or of preparing a capsule formulation in storage-stable form. A formulation with these properties would permit the use, for example, on the skin or on the surface of leaves.

Conventional formulations of liquids, such as solutions, emulsions or double emulsions, generally release the active ingredient very rapidly following application of a thin film to a surface. Emulsions and double emulsions are destroyed by the vaporizing dispersion medium and the capillary forces which then arise, and the release kinetics as in the case of simple solutions are only determined by the vapour pressure of the active ingredient solution. Formulations which are based on mechanically stable capsules and have been prepared in a conventional manner, e.g. by interfacial polymerization or spray-drying, are generally so stable that they remain intact even in the dry state and can release the active ingredient either by very slow diffusion through the capsule wall or only if the capsule has been mechanically destroyed. In addition, active-ingredient-containing capsules are often dispersed prior to use in a liquid phase (e.g. in the field of crop protection) or are suspended in a considerable excess of a liquid phase during preparation. Months, sometimes years then often pass until they are used. In the field of pharmacy, shelf-lives of from three to five years are often required. Due to the mostly desired semi-permeability of the capsule walls, the active ingredient consequently diffuses until the dispersant is saturated into the outer phase, meaning that in certain circumstances only a small amount of active ingredient remains in the capsules and/or, however, a concentration in the outer phase is reached which produces undesired secondary effects (e.g. toxicity), or the optimum effective concentration for the initial effect is exceeded. In addition, in some cases, there is the need to provide two or more active ingredients to achieve a broad activity spectrum against biological pests/parasites in a formulation which cannot normally be formulated in the necessary concentration in a presentation as a result, for example, of differing solubilities in toxicologically acceptable solvents or as the result, for example, of chemical incompatibility.

An often used way of developing formulations with delayed or controlled release behaviour is the use of microcapsules. These can be prepared conventionally in various ways and can consist either of capsules with liquid or solid contents. These processes are interfacial polymerization, interfacial precipitation reactions, complex and simple coacervation, and complex emulsification (double and microemulsions). These processes are generally known and described in numerous publications (see e.g. T. Kondo, Journal of Oleo Science 50, 1 (2001); T. Kondo, Journal of Oleo Science 50, 81 (2001) or C. Thies, Encycl. Polym. Sci. Eng. 9, 724 (1987)).

A relatively new method of encapsulating solid or liquid dispersion particles is the layer-by-layer growth of a mantle membrane which is produced by alternate deposition of cationic and anionic polyelectrolytes, where appropriate with incorporation of charged nanoparticles (cf.: G. B. Sukhorukov et al. Colloids and Surfaces A, 137, 253-266 (1998); patent WO 9947252, WO 9947253). In an alternative variant of the described process, it is possible, in a coacervation process, to carry out the precipitation of polyelectrolytes on the interfaces of pre-emulsified liquid droplets or of solid particles also by the one-step process. In this connection, the polyanions and polycations, which are present together in solution, are precipitated out directly onto the surfaces by shifting the pH and/or salts content (cf. WO 2002009864 Encapsulation of liquid template particles using amphiphilic polyelectrolytes, DE 10050382, WO 2002031092 Method for the inclusion of perfume oil in washing and cleaning agents or cosmetics). The disadvantage of the described process is that, following removal of the dispersant, e.g. following application to a surface, the coated emulsion droplets are often not stable, but rapidly deliquesce.

The object of the invention is to develop a new process which makes it possible to provide a formulation with the above mentioned desired properties, i.e. following application of the formulation to a surface, supplies the active ingredient in a defined concentration in the outer phase (dispersant) to achieve a desired initial effect, permits a controlled release rate from the capsule to establish a delayed, long-lasting effect, and ensures storage stability of such a capsule suspension.

The above object was achieved by a formulation consisting of a plurality of phases in which the active ingredient may be present in each phase in a different concentration. Release from the various phases proceeds at different rates, meaning that, by varying the amounts of active ingredient used in each case and the type of solvent/dispersant used, it is possible to vary the kinetics and the amount of active ingredient released overall.

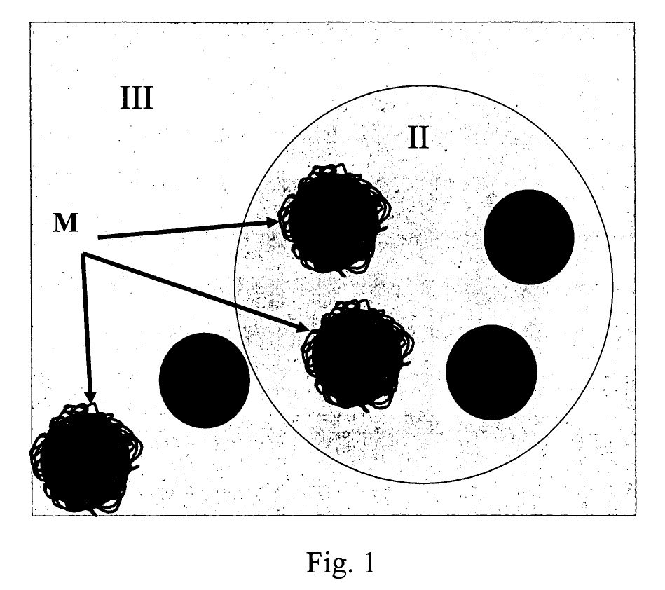

The invention provides an active-ingredient-containing formulation with a plurality of active-ingredient-containing phases which is characterized in that the formulation has a first innermost finely divided phase (I) which consists of active ingredient or active ingredient solution, of which preferably some phase particles are surrounded with a barrier mantle (M), and that the formulation has a second, middle phase (II) which serves as dispersant for the first, inner phase (I) and in which active ingredient may likewise be dissolved, and that the formulation has a third outer phase (III) which serves as dispersant for the second middle phase and in which active ingredient may in turn be present in dissolved form and/or in the form of solid particles, which again may be surrounded with a barrier mantle. This principle is shown diagrammatically in FIG. 1.

In a special case of the invention, the phases (I) and (II) are not dispersed as described within one another and subsequently in the outer phase (III), but form a 3-phase layer system in the sense that the middle phase (II) covers the inner phase (I), and the phase (II) is itself in turn covered by phase (III). This variant of the invention can be particularly advantageous if diffusion of the active ingredient from the innermost phase takes place rapidly and slow release can only be achieved by minimizing the phase interfaces.

Also described is the solidification of the intermediate phase (II) in order to mechanically stabilize the dispersion from inner phase (I) following application to a surface, and thus to obtain delayed release of the active ingredient in the inner phase (I) and/or the intermediate phase (II). The invention further provides that, in the manner described, it is possible to prepare a plurality of biologically effective active ingredients in the different phases in varying concentrations and with a release rate which is controlled in each case in a single formulation.

Preference is given to a formulation which is characterized in that the barrier mantle in the various phases is a microcapsule. In the text below, microcapsule is understood as meaning either a capsule with a solid polymer wall, or a capsule whose wall consists of a relatively thin polymer layer or membrane which can be produced, for example, by coacervation.

The microcapsule of the barrier mantle is particularly preferably based on a polymer.

In a preferred formulation, the outer third phase (III) is an oil phase with limited solubility for the active ingredient or active ingredients, preferably of silicone oil or native oils—e.g. castor oil—or perfluorinated organic compounds. This outer phase can additionally comprise dispersion auxiliaries (surfactants) or thickeners (e.g. Aerosils, polymers).

In a further preferred formulation, the second, middle phase (II) is based on a thickened phase containing polymer or solid particles, e.g. a gelatin solution.

In a further particularly preferred formulation, the second middle phase (II) consists of a polymer solution or particle dispersion which is thermoreversibly gellable, i.e. is liquid at the temperatures of the separation and/or application, and semisolid or solid at the temperatures during storage and/or use. In addition, the active ingredient/the active ingredients preferably exhibit lower solubility in this middle phase.

The innermost phase (I) consists of the pure active ingredient or a solution of active ingredient. This phase can be present as an emulsion droplet stabilized by a with surfactants, or a solid or semisolid dispersion particle. The inner phase can likewise consist of a microcapsule which comprises the liquid, solid or semisolid active ingredient. The capsule wall of the microcapsule (M) can be prepared, for example, by complex coacervation and represents a first barrier for the active ingredient. These droplets, dispersion particles or microcapsules of phase (I) are then introduced into the second liquid, thickened or semisolid matrix phase (II), which represents the second barrier. The thickened, semisolid matrix phase at the same time produces the required mechanical stability, which permits application of the formulation in the form of a film without immediate destruction of the matrix phase.

Finally, these multicapsules are again dispersed in the outer phase (III) which can consist of a defined solution of the active ingredient and/or the active ingredients or of a phase which has only a very low saturation solubility or no solubility at all for the active ingredient/the active ingredients. In this outer phase saturated with active ingredient, the active ingredient/the active ingredients can additionally also be present in dispersed form in the form of emulsion droplets.

The particle sizes of the innermost phase (I) can be varied through the amount of substances used and the type of dispersion. They are typically in the order of magnitude 1-10 μm. The size of the particles from emulsified intermediate phase (II) and inner phase (I) is typically in the order of magnitude of 10-500 μm.

In such a multiphase system, the active ingredient/the active ingredients is/are thus present in different phases and the release of the active ingredient/of the active ingredients is determined by the kinetics of the diffusion into the phases and by the phase boundaries, and by the physical boundary solubility(ies) of the active ingredient/of the active ingredients in the phases. Since the amounts of active ingredient used in the various phases are variable, the desired release profile can be adjusted in a targeted manner. The disadvantage of the customary capsule formulations, the emptying of the capsules by continuous release into the outer phase during storage is likewise thereby overcome.

The invention further provides the use of the formulation according to the invention for the stagewise delayed release of active ingredients.

Release Behaviour of the Multiphase Systems

In summary, the active ingredient/the active ingredients can thus be released from the following phases:
a) rapid release to ensure immediate effect (knock-down effect): from the outer phase (III) which consists of the pure active ingredient, a solution of the active ingredient or an emulsion.
b) delayed release: active ingredient is present as a molecular solution or as droplets in a solid or semisolid matrix (II), which additionally ensures mechanical stability when applied in the form of a film and whose saturation solubility determines the diffusion profile of the active ingredient.
c) slowed release: active ingredient is in the form of microcapsule (I) in the solid or semisolid matrix (II) and must therefore also penetrate a further barrier

mantle.

A further advantage of the multicapsule system is the possibility of also preparing formulations which comprise more than one active substance by introducing various active ingredients into the various phases, the required release profile of which can then in each case be adjusted separately. In the case of very different dissolution behaviour in the individual phases, chemically or physically incompatible active ingredients can thus also be formulated together.

A further advantage of the multiplecapsule system consists in establishing the concentration of free active ingredient in the outer phase (III) by choosing suitable solvents and solvent mixtures, in the sense that that in the outer phase an appropriate saturation solubility is established.

In principle, the multiplecapsule system can also be used to release active ingredients into a liquid environment.

Feed Materials

Formulations which are based on the described invention can preferably be used in the field of dermal formulations on humans and animals, and also in the field of crop protection. Consequently, preference is given to using auxiliaries and solvents which are toxicologically safe and are authorized/or in principle classified as licensable by the competent authorities.

I) Solvents

Inner Phase (I):

Liquid or solid active ingredient or solutions of these active ingredients in pharmaceutically and environmentally acceptable, nontoxic oils with low polarity (dielectric constant) or solutions in solid matrix phases. These solvents are, for example, but not exclusively:

medium-chain triglycerides (e.g. Miglyol 810, 812); native oils (e.g. castor oil, sesame oil, peanut oil), partially hydrolysed fats or reaction products of such partially hydrolysed fats with low degrees of ethoxylation (e.g. Labrafil, Gelucire); hydrophobic esters of native fatty acids (e.g. isopropyl myristate); hydrophobic solvents of higher polarity (e.g. triethyl citrate, triacetin); semisolid or solid matrix-forming systems into which the active ingredient is incorporated in molecularly disperse form and which are liquid during preparation and solid during storage and/or application (e.g. fats, shellac, polyethylene glycols PEG 1000, 1500, 3000)

Matrix Phase (II):

Pharmaceutically or environmentally acceptable, nontoxic, relatively hydrophilic systems in which the active ingredient/the active ingredients and the solvents of the inner and outer phases are insoluble or soluble only to a very limited degree and which can in turn act as solvent for the thickening additives (polymers, hydrotalcites). Such solvents are, for example, but not exclusively:

water, mixtures of water with other hydrophilic solvents, hydrophilic solvents of suitable polarity (dielectric constant) (e.g. propylene glycol, ethanol, ethanediol, glycerol, polyethylene glycols of low chain length PEG 200, 300, 400)

The middle phase can also comprise surfactants for the initial stabilization of the inner phase during preparation, for example, but not exclusively: ionic surfactants (dodecyl sulphate Na salt (SDS)), cationic surfactants (cetyltrimethylammonium chloride) or natural or synthetically produced polyelectrolytes (gelatin, polystyrene sulphonate) or polymeric nonionic dispersants (polyvinyl alcohol-polyvinyl acetate copolymers, e.g. Moviols)

Outer Dispersion Phase (III):

Liquid or solid active ingredients or solutions of these active ingredients in pharmaceutically and environmentally acceptable, nontoxic oils with low polarity (dielectric constants). Such solvents are, for example, but not exclusively:
medium-chain triglycerides (e.g. Miglyol 810, 812); native oils (e.g. castor oil, sesame oil, peanut oil), partially hydrolysed fats or reaction products of such partially hydrolysed fats with low degrees of ethoxylation (e.g. Labrafil, Gelucire); hydrophobic esters of native fatty acids (e.g. isopropyl myristate); hydrophobic solvents of higher polarity (e.g. triethyl citrate, triacetin);
silicone oils or perfluorinated solvents which have a low dissolution capacity for the hydrophilic middle phase and likewise for the oils of the inner phase and for the active ingredients, e.g. dimethyl-polysiloxanes of varying chain length. (e.g. Dow Corning Q7-9120 Silicon Fluid Series 20, 100, 350, 1000), cSt, higher cyclic dimethyl-oligosiloxanes (e.g. dodecamethylcyclohexasiloxane), perfluorinated alkanes or perfluorinated polyethylene oxides;
preferably those silicone oils or organic oils which have a low viscosity, high vapour pressure and good spreading behaviour such that, following the use on skin or plants, they ensure rapid distribution of the matrix phase particles and then they volatilize without leaving a residue in order, for example, to avoid greasy residues, e.g. cyclic polysiloxanes octamethylcyclotetrasiloxane D4, decamethylcyclo-pentasiloxane D5, short-chain linear oligodimethylsiloxanes (e.g. DOW Corning Q7-9180 Silicon Fluid Series 0.65 cSt hexamethyl-disiloxane, 1 cSt octamethyltrisiloxane, 5 cSt). The outer phase can also additionally comprise surfactants and dispersants for stabilizing the matrix phase, e.g., but not exclusively: polyethylene oxide-poly-propylene oxide-polyethylene oxide copolymers (e.g. Pluronics, poloxamers), ethoxylated carboxylic esters or alkyl ethers (e.g. Cremophors), polyethylene oxide-modified polydimethylsiloxanes (e.g. DOW Corning DC 5225C, DC 3225C, Emulsifier 10)
   II) Polymeric Feed Materials and Thickeners:
For the initial stabilization of the inner phase in the middle matrix phase and for producing the first diffusion-controlling membrane wall (M), cationic and anionic polyelectrolytes can preferably be used, for example but not exclusively: polystyrene sulphonate (PSS), polyallylamine hydrochloride (PAH), polydiallyldimethylammonium chloride, gelatin, carboxymethylcellulose, xanthan.
For stabilizing the phases and adjusting the rate of diffusion of the active ingredients through the phases and phase boundaries, preference is given to using those polymers and inorganic particles which are dispersible or soluble in the phases with suitable dielectric constant. Solubility of polymers is understood here as meaning that the solvent of the particular phase, in particular of the medium matrix phase (II), represents a thermodynamically good solvent in the sense of the Flory-Huggins theory (χ<0.5) for the polymer and this thus has a gel-forming effect. Particular preference here is given to those polymers which have thermoreversibly thickening properties in the particular solvent. Alternatively, those polymers or surface-active auxiliaries can be used which exhibit a thermally induced liquid-liquid crystalline/semisolid phase transition between preparation conditions and storage conditions.
For the more hydrophilic phase (II) mention can thus be made, for example, but not exclusively, of: polymers and polyelectrolytes derived from natural substances (e.g. hydrocolloids: gelatin, xanthan, pectins, carrageenan, carboxymethylcellulose), surface-active feed substances which form LC phases (e.g. polyethylene oxide-poly-propylene oxide-polyethylene oxide copolymers pluronics/poloxamers, polylactide-co-glycolide block copolymers with polyethylene glycol), synthetically prepared polymers (e.g. partially hydrolysed polyvinyl acetates of suitable degree of hydrolysis: Mowiol 3-83, 10-74, poly-N-isopropylacrylamide NIPAAM, and simply thickening polymers, such as polyvinyl alcohol, polyacrylic acid and polyacrylic ester copolymers thereof, carboxymethyl-cellulose, hydroxypropylmethylcellulose, hydroxyethylcellulose, inorganic minerals (e.g. hectorites, silica).
For the inner phase (I) it is possible to use those polymers and feed materials which are soluble or dispersible in the solvents used of suitable dielectric constant, for example, but not exclusively: polyacrylamides, polyacrylic esters, N-isopropylacrylamide, polyvinyl acetates and vinyl acetate-vinyl alcohol copolymers of low degree of hydrolysis (e.g. Polyviol 45/450), ethylcelluloses, methylcelluloses, inorganic thickeners (silica, aerosil), or those feed materials which can themselves act as a solid matrix phase for the active ingredient, e.g. feed materials obtained from natural products (shellac, beeswax) or polyethylene oxides of higher molecular weight (e.g. PEG 1000, 1500, 3000)
For the outer phase (III) it is in principle possible to use the same feed materials for the stabilization which are chosen according to the criteria for the inner phase (I), but are generally dispensed with when the spreadability of these formulations on the surfaces following application is a decisive criterion.
   III) Active Ingredients

In the multiphase systems according to the invention it is possible to incorporate a plurality of encapsulated active ingredients with varying properties and release profiles separately from one another. Nonexclusive examples are:

Active ingredients with a high potential for skin irritation (e.g. pyrethroids flumethrin, permethrin, cyfluthrin), insecticidal systemic active ingredients (e.g. imidacloprid), readily volatilizing agents, i.e. e.g. repellents (e.g. N,N-diethyl-m-toluamide DEET, 2-(2-hydroxyethyl)-1-methylpropyl 1-piperidinecarboxylate KBR 3023) or attractants/pheromones, (e.g. 8,10-E,E-dodecadienol, codlemone), care active ingredients (e.g. vitamins), antiinflammatory active ingredients (cortisone) or fungicidal active ingredients (e.g. clotrimazole).

Producing the Multiphase Systems

The preparation of the multiphase system described here can generally be described by the following steps:
1.) Emulsification or Dispersion of the Innermost Phase (I) in a Continuous Phase
   Known standard dispersion and emulsification processes can be used to emulsify or disperse the innermost phase (I) (e.g. stirrers, ultrasound sources, Ultra-Turrax, membrane emulsion). Ionic or nonionic surfactants or polymers are used for stabilizing the innermost phase (I). The continuous phase can represent the intermediate phase or matrix phase (II) and only achieve the ultimate composition of the intermediate phase (II) in a later step, e.g. by adding soluble polymers.
2.) Encapsulation of the Emulsified or Dispersed Innermost Phase (I)
   All or some of the innermost phase (I) is encapsulated by one of the known encapsulation methods, such as interfacial polymerization, interfacial precipitation reactions, complex or simple coacervation or polyelectrolyte precipitation. An alternative way is the encapsulation of the innermost phase (I) outside of the continuous phase before step 1 by a known method, such as, for example, spray-drying.
3.) Emulsification of the Resulting Emulsion Dispersion or Microcapsule Dispersion in an Outer Phase (III)
   The resulting emulsion or suspension is then emulsified in a further dispersion step in the outer phase (III). Customary dispersion or emulsification methods and nonionic or ionic surfactants or polymeric stabilizers are again used for this.
4.) Solidification of the Intermediate Phase (II) During or after the Second Emulsification Step (Step 3)
   In the last step, the intermediate phase is solidified during or after step 3. The solidification can be induced, for example, by changing the temperature, pH or ionic strength in the intermediate phase (II) and of the outer phase (III).

Through simple modification of this preparation process it is possible to prepare a multiphase formulation which is not in the form of a complex emulsion, but in the form of a simple layer system of a plurality of phases.

The advantages of the invention described here are summarized as follows:
a) The solidification of the intermediate phase of a double emulsion directly after or during the second emulsification step. As a result of this, the particles of the double emulsion achieve higher mechanical stability and remain intact for longer during or after the drying operation than, for example, non-solidified double emulsions.
b) By choosing a solvent which spreads well on a surface (e.g. plant cuticula or human or animal skin) for the outer phase which additionally has a defined limited solubility for the active ingredient/the active ingredients to safeguard an initial effect, the multicapsule consisting of phase (I) and (II) can be distributed rapidly. In addition, it is often desired for this outer carrier phase to volatilize rapidly following application. The property mentioned under (a) ensures that the multicapsules on the surface also then remain stable and release the active ingredient/the active ingredients in a delayed and controlled manner.
c) The combination of double emulsion and microcapsules, which leads to a system with a plurality of barriers for the active ingredient. This permits the distribution of the active ingredient substance in different phases with varying barrier properties and thus control of the release.
d) The targeted adjustment of the solubility of the active ingredient in the inner, middle and outer phase through the choice of suitable materials, as a result of which diffusion of the active ingredient through the various phases and thus the release profile can be controlled.
e) The overcoming of the storage problem: capsules through which the active ingredient can in principle penetrate are not emptied even after storage for one year since the active ingredient has only limited solubility, or is not soluble at all, in the outer phase.

The invention is illustrated in more detail below, for example, by reference to the figures.

These show:

FIG. 1 a scheme for building up the active ingredient formulation according to the invention

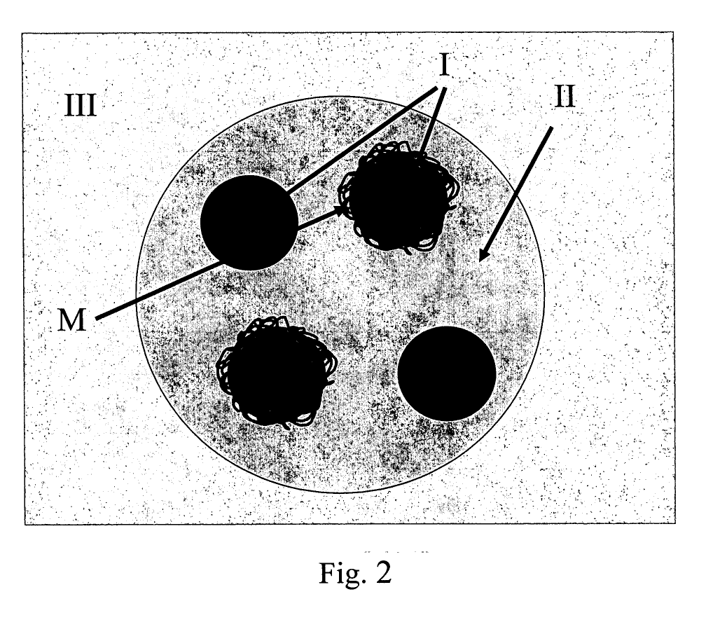

FIG. 2 a scheme for an alternative build-up of the active ingredient formulation

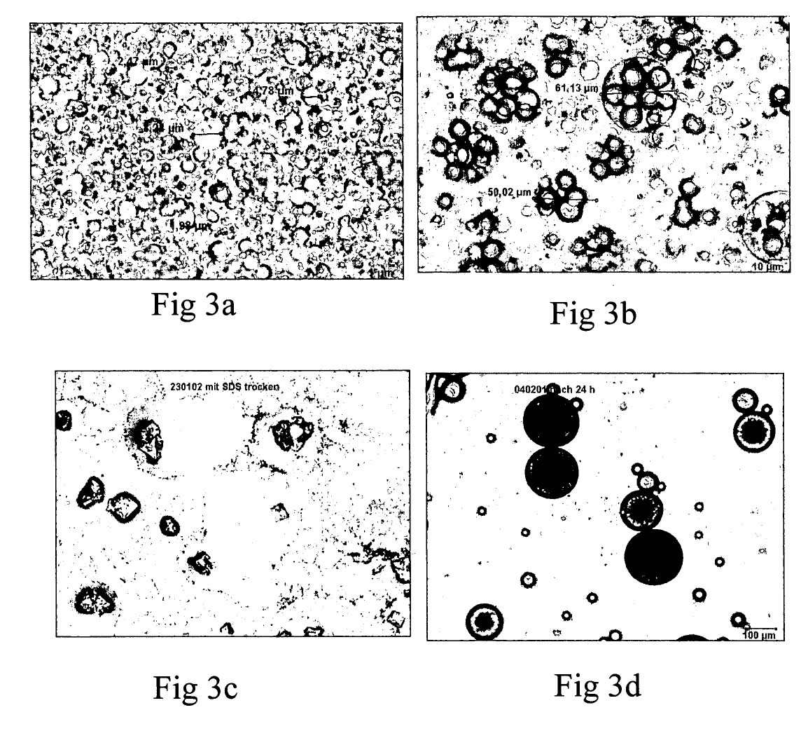

FIG. 3a a micrograph of the primary capsule

FIG. 3b a micrograph of the multicapsule

FIG. 3c a micrograph of the formulation according to FIG. 3a following application to a surface

FIG. 3d a micrograph of the formulation according to FIG. 3b following application to a surface

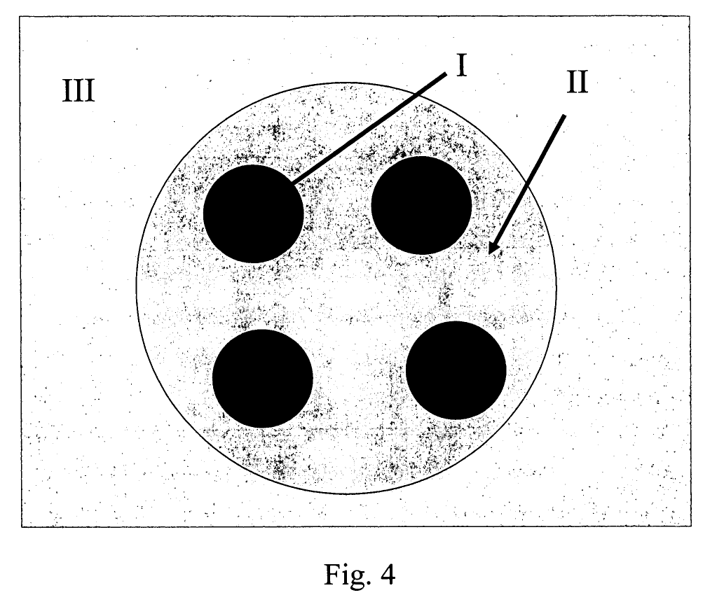

FIG. 4 the scheme for building up a further variant of the active ingredient formulation

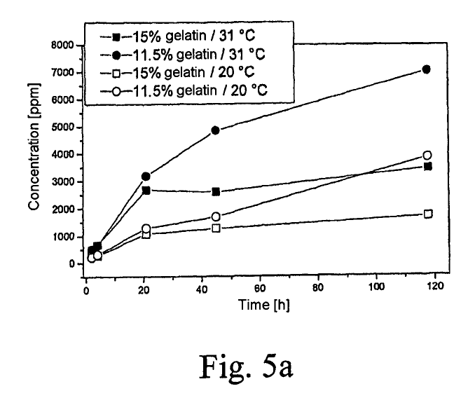

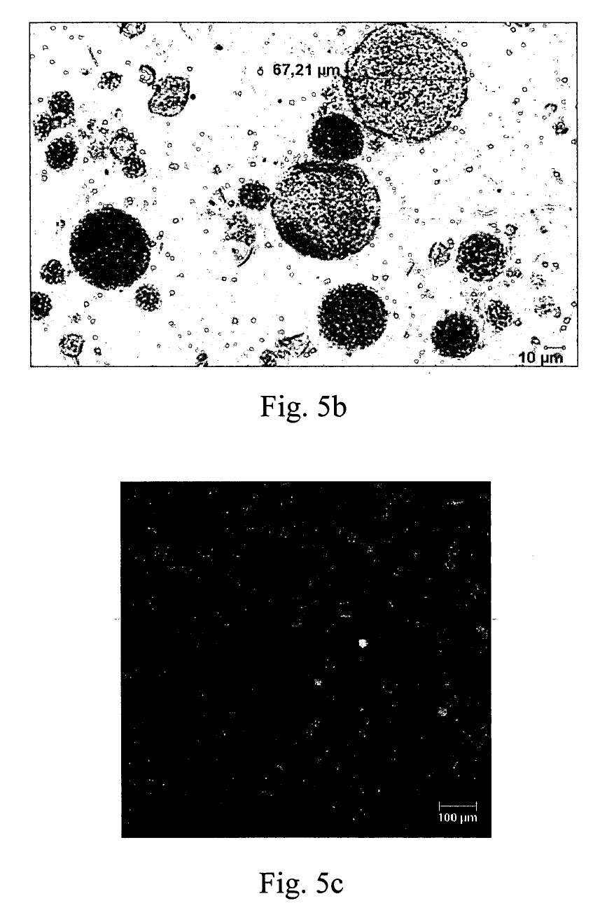

FIG. 5a a diagram for the release of flumethrin as a function of time

FIG. 5b a micrograph of a flumethrin formulation

FIG. 5c a micrograph of the flumethrin formulation according to FIG. 5b after ageing

### EXAMPLES

### Example 1

As an example, this principle was applied to the formulation of KBR 3023. This example is shown diagrammatically in FIG. 2. KBR 3023 is a liquid active ingredient which is only slightly soluble in water and also is soluble in silicone oils only to a limited degree. In the example formulation, the outer phase consists of a KBR/silicone oil mixture (III). The middle matrix phase consists of an aqueous gelatin solution (II) which is liquid during the preparation since the operating temperature was above the gel temperature. After cooling to room temperature, this matrix is semisolid or solid. KBR 3023 droplets form the inner phase (I) and are in the form of microcapsules with a polymeric mantle which was prepared by complex coacervation of PSS and PAH (M), in the gelatin matrix.

The following preparation procedure is one example of such a formulation:

Step 1:

Dissolve 0.038 g of sodium dodecyl sulphate (SDS) in 5 g of water (saturated with KBR 3023),

Addition of 2.5 g of KBR 3023. Dispersion using Ultra-Turrax (UT).

Step 2:

Addition of 5 g of aqueous PSS solution saturated with KBR (conc.: 2.06 g/100 g). With the Ultra-Turrax running, dropwise addition of 5 g of aqueous PAH solution saturated with KBR (conc. 0.96 g/100 g).

Step 3:

Careful mixing of 0.5 g of warm 25% strength gelatin solution with 0.5 g of primary emulsion. This mixture is added to 2 g of warm silicone oil phase. The silicone oil phase consists of a linear silicone oil DC 5 (Dow Coming, 0.3 g of liquid KBR+0.2 g of emulsifier 5225 C+1.5 g of oil). Dispersion with UT with subsequent rapid cooling by an ice bath. After-stirring time of about 15 min.

FIG. 3a shows the primary capsules produced by step 2. FIG. 3b shows the multicapsule with solidified intermediate phase.

If the emulsions from FIG. 3a are applied to a surface, then the primary particles are destroyed after a few minutes and the KBR 3023 is in the form of a free film (FIG. 3c). If, however, the emulsion from FIG. 3b is applied as a film, then the multi-capsules are still stable even after 24 h (FIG. 3d). This demonstrates one of the decisive advantages of the invention described here.

Furthermore, the saturation of the outer phase (III) and of the intermediate phase (II) with KBR 3023 prevents the diffusion of KBR 3023 from the inner phase (I) during storage of the formulation.

By simply modifying the above formulation, KBR droplets without a mantle can be dispersed alternatively or additionally both in the gelatin matrix (II) and in the outer phase (III). Likewise, KBR droplets without a mantle can alternatively or additionally be emulsified in the outer phase.

### Example 2

As a further example, this formulation principle was applied for the formulation of a dermal active ingredient formulation for controlling ticks and fleas in veterinary medicine. The formulation is shown diagrammatically in FIG. 4. Here, the active ingredient (flumethrin) is dissolved in an oily phase (I), which is emulsified in an aqueous gelatin solution above the gel temperature (II), and this emulsion is itself dispersed into an outer silicone oil phase (III). After cooling to below the gel point, the gelatin matrix solidifies. The silicone oil phase is chosen such that the physical limiting solubility in the outer phase corresponds exactly to the concentration which is favourable for the active ingredient to be effective directly after application. During storage after preparation, the active ingredient flumethrin thus accumulates in the outer phase until this optimal concentration is reached. Over the course of time, release from the innermost phase is delayed by the gelatin matrix since the skin temperature is below the softening point of the gelatin gel. The outer phase additionally also comprises a further dispersed active ingredient (imidacloprid) in order to ensure a thorough insecticidal action. As a result of its spreading ability, the silicone oil phase aids the rapid distribution of the dispersed particles of the matrix phase and of the second active ingredient on the skin. The use of a silicone oil with a low vapour pressure additionally ensures that this phase evaporates rapidly after spreading and thus does not leave behind a greasy impression.

The following preparation procedure is one example of such a formulation:

Preparation of the Various Phases:

Inner Phase (I):

1.89 g of flumethrin and 0.108 g of Lipoid S100 are weighed into 0.702 g of Miglyol and dissolved at elevated temperature.

Intermediate Phase (II):

0.063 g of taurocholic acid (TCA), 0.0945 g of methyl parahydroxybenzoate (PHB) and 0.7245 g of gelatin are weighed one after the other into 0.725 g of water. The mixture is stirred at a temperature above the gel temperature using a magnetic stirrer until the gelatin has dissolved.

Outer Oil Phase (III):

0.8925 g of Na stearate and 6 g of NTN (imidacloprid) are added to 44.108 g of silicone oil (Fluka DC 200 20 mPas). By means of UT (20 500 rpm), the suspension is homogenized and simultaneously heated to about 40-60° C.

Preparation of the Formulation:

The about 60-80° C.-hot inner oil phase (I) is added dropwise to the warm gelatin phase (II). Here, the speed of the UT must be increased in stages such that maximum dispersion always takes place. The temperature is controlled at the same time. The temperature is kept at about 50-60° C. by means of a water bath. Addition time about 4 min, after-stirring time about 6 min.

Then, with stirring using the UT, the warm primary emulsion is added to the about 50° C.-hot outer oil phase. The temperature is kept at about 45-50° C. by means of a water bath. Addition time about 4 min, after-stirring time about 2 min. The water bath is then replaced with ice/NaCl. The mixture is further stirred during this cooling to RT.

FIG. 5a shows the release of flumethrin into the outer phase (III) as a function of time. It can be seen that by varying the gelatin concentration in the intermediate phase (II), it is possible to vary considerably the release rate and amount. Similarly, by changing the temperature it is possible to vary the release rate and amount.

FIG. 5b shows a micrograph of the formulation. Particles are evident which consist of the intermediate phase (II) and the flumethrin droplets (I) emulsified therein.

FIG. 5c shows a formulation stored for 30 days at 40° C. in fluorescent light. The pale areas consist of flumethrin (I) and it can be seen that the active ingredient, even after storage, has not diffused from the inner phase (I) and the intermediate phase (II) into the outer phase (III). Otherwise the continuous outer phase (III) would also appear light in this photograph.

In order to make the multicapsules more visible, a formulation without NTN was used in the outer phase (III) for these photographs.

### Example 3

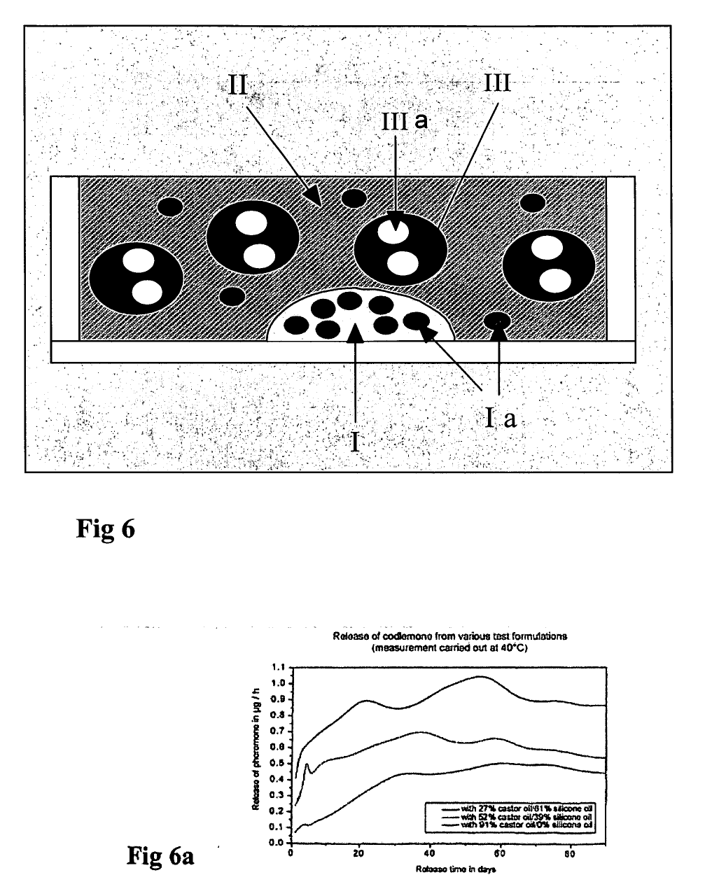

Finally, as a third example, the development of an Attract&Kill formulation with a particularly long-lasting constant release of the attractant for the cultivation of fruit can be specified. This example is shown diagrammatically in FIG. 6. Here, an attractant (pheromone codlemone, Ia) was fused in a suitable concentration into a highly viscous to solid matrix (beeswax, relatively high molecular weight polyethylene glycols, shellac) (I). This formulation represents one of the described special cases in the sense that the phases (II)—a silicone oil phase—and (III)—a castor oil phase—have been charged together as a 2-phase system over a highly viscous to solid depot phase (I). Here, the silicone oil phase (II) in turn comprises further attractant (Ia) for immediate release. Castor oil as the third phase (III) serves to control the release rate and at the same time functions as a solvent for a second active ingredient (cyfluthrin, IIIa). Additional control can take place via the size of the interface between the phases (I) and (II/III) through control of the diffusion. To further reduce the release rate, it is also possible to dispense completely with phase II. The formulation can be applied to the plants either using a suitable applicator as drops or poured into a moulded vessel. In this case, the outer phase is also enriched with a thickener (Aerosil) in order to prevent the formulation from immediately running off. The diffusion kinetics of the attractant from the inner phase ensure a constant concentration profile in the formulation, meaning that release from the outer phase into the air in the required concentration can be maintained over more than 100 days.

3 examples are given below in which the volume ratios of the phases (II) and (III) have been varied. The preparation of the reservoir (step 1), like the preparation of the formulations, are identical in all cases.

Preparation of the Various Phases:

Step 1 (Reservoir):

Liquefy 0.285 g of beeswax, addition of 15 mg of codlemone. Dispersion with magnetic stirrer. This warm mixture is added dropwise to e.g. a crimp cap N20 and left to solidify.

Step 2 (Phase 2):

a) Add 1.22 mg of pheromone codlemone to 1.27 g of silicone oil (Fluka DC 200 1020 mPas). Dispersion with magnetic stirrer.
b) Add 1.22 mg of pheromone codlemone to 0.77 g of silicone oil (Fluka DC 200 1020 mPas). Dispersion with magnetic stirrer
c) no silicone oil phase
   Step 3 (Phase 3):
a) Heat 0.548 g of castor oil (27%), with stirring add 80 mg of cyfluthrin. At elevated temperature, stir until a clear solution has formed.
b) Heat 1.04 g of castor oil (52%), with stirring add 80 mg of cyfluthrin. At elevated temperature, stir until a clear solution has formed.
c) Heat 1.82 g of castor oil (91%), with stirring add 80 mg of cyfluthrin. At elevated temperature, stir until a clear solution has formed. 1.22 mg of codlemone is added to the solution.
   Preparation of the Formulations:

Warm phase 3 was dispersed into phase 2. After homogenization, 98 mg of Aerosil 150 are added. The solid is added in portions with stirring. The crimp cap filled with the reservoir is then topped up with the pasty phase homogeneously and without trapped air.

The release rates achieved in the gas phase depend on the mixing ratio of the silicone phase (II) and of the castor oil phase (III). Through variation, the kinetics can be adapted to the desired conditions and/or requirements. FIG. 6a shows a measurement series in which the mixing ratios between castor oil and silicone oil were continuously changed.

1. Active-ingredient-containing formulation comprising a plurality of active-ingredient-containing phases wherein the formulation comprises a first innermost finely divided phase (I) which consists of active ingredient or active ingredient solution, wherein at least some phase particles optionally are surrounded with a barrier mantle (M), and that the formulation has a second, middle phase (II) which serves as dispersant for the first, inner phase (I) and in which active ingredient may likewise be dissolved, and a third outer phase (III) which serves as dispersant for the second middle phase and in which active ingredient may in turn be present in dissolved form and/or in the form of solid particles, which optionally are surrounded with a barrier mantle.

2. Formulation according to claim 1, wherein the intermediate phase (II) acts as mechanical stabilization of the innermost phase (I).

3. Formulation according to claim 1, wherein a plurality of biologically effective active ingredients in the various phases are combined in varying concentrations and with a release rate which is controlled in each case in a single formulation.

4. Formulation according to claim 1, wherein the formulation limits the release of the active ingredient from the innermost phase by choosing a suitable solvent for the outer phase.

5. Formulation according to claim 1, wherein the barrier mantle in the various phases is a microcapsule.

6. Formulation according to claim 5, wherein the microcapsule of the barrier mantle is based on a polymer.

7. Formulation according to claim 1, wherein the outer third phase is an oil phase.

8. Formulation according to claim 1, wherein the inner second phase is based on gelatin.

9. A method for the controlled release of an active ingredient in a patient comprising administering to said patient a formulation according to claim 1.
